(19) United States
(12) Patent Application Publication
(10) Pub. No.: US 2010/0228258 A1
(43) Pub. Date: Sep. 9, 2010

(54) INTRAMEDULLARY NAIL TARGETING DEVICE

(76) Inventor: Alfred A. Durham, Roanoke, VA (US)

Correspondence Address: Intellectual Property Dept.;Dewitt Ross & Stevens SC, 2 East Mifflin Street, Suite 600, Madison, WI, 53703-2865, US

(21) Appl. No.: 12/552,726

(22) Filed: Sep. 2, 2009

Related U.S. Application Data
(63) Continuation-in-part of application No. 10/679,166, filed on Oct. 3, 2003, now Pat. No. 7,753,913.
(60) Provisional application No. 61/190,709, filed on Sep. 2, 2008.

(51) Int. Cl. A61B 17/56

(52) U.S. Cl. 606/96

(57) ABSTRACT

An intramedullary nail targeting device for detecting the precise location and position of an opening in an intramedullary nail in a bone or similar object within a body of tissue is described. The device includes a body with a handle end and a drill sleeve end, an activation button, a drill sleeve connected to the drill sleeve end and having a first proximal end and a second distal end. The drill sleeve has a length necessary to extend through the depth of the tissue and includes a sensor foot and a drill guide. The sensor foot includes a sensor array for sensing the opening in the intramedullary nail when the sensor foot is placed on or near the surface of the bone.

### REFERENCE TO RELATED APPLICATIONS

This is a continuation-in-part to U.S. patent application Ser. No. 10/679,166, in the name of Szakelyhidi, Jr. et al., entitled “Magnetic Targeting Device” filed Oct. 3, 2003 (hereinafter “Szakelyhidi et al.”) and claims priority to U.S. Provisional Patent Application Ser. No. 61/190,709, filed Sep. 2, 2008, both of which are incorporated herein by reference in their entirety.

### FIELD OF THE INVENTION

The present invention is directed to a targeting device in general and specifically relates to an intramedullary nail (“IMN”) targeting device and method for positioning locking screws for intramedullary nails.

### DESCRIPTION OF THE PRIOR ART

The use of magnetic targeting to locate hidden holes or openings in orthopedic hardware has been tried in many forms. However, the distances involved make sensing the magnetic fields difficult. Even the fields of the strongest magnets diminish to that of the earth's magnetic field at distance of about 10 cm.

The earliest successful magnetic targeting was accomplished by Durham et al. and was described in a succession of patents covering a mechanical magnetic targeting system using a mechanically balanced cannulated magnet (U.S. Pat. Nos. 5,049,151; 5,514,145; 5,703,375; and 6,162,228). Hollstien et al. (U.S. Pat. No. 5,411,503) followed with an electrically based system of stacked flux finders connected to a PC display.

The magnetic targeting devices to date target a magnet to accurately position a drill bit for insertion in an opening in intramedullary nails. However, these devices operate at the level of the skin, and the magnet may not be strong enough to accurately position the drill bit. As a result, all of these systems are subject to interference and slow response time and are yet to be practical in surgical use. The key component lacking in these systems is a way to target the surface of the bone where the magnetic field strengths are the highest.

### SUMMARY OF THE INVENTION

The present invention solves the issue of diminished magnetic strength by placing the magnetic sensors of the magnetic targeting device directly on the bone. This is accomplished by affixing the magnetic sensors directly on the drill bit cannula at the area of the IMN opening. The configuration resembles a foot wherein the system of sensors is in the area of the toe portion.

The present invention is specifically directed to an intramedullary nail targeting device for detecting the precise location and position of an opening in an intramedullary nail in a bone or similar object within a body of tissue having a depth, comprising a body including a handle end, a drill sleeve end, a power source and processing circuits for sensing the correct orientation of the device with respect to the bone; an activation button; and an extended drill sleeve connected to the drill sleeve end and having a first proximal end and a second distal end, wherein the drill sleeve has a length necessary to extend through the depth of the tissue. The drill sleeve includes a sensor foot at the distal end of the drill sleeve, a drill guide extending from the proximal end of the drill sleeve to the distal end, and a sensor array within the sensor foot for sensing the opening in the intramedullary nail when the sensor foot is placed on or near the surface of the bone. The intramedullary nail targeting device further includes display means to determine the correct orientation of the device with respect to the bone when the sensor foot is placed on or near the surface of the bone.

The present invention is also directed to a system for detecting the precise location and position of an opening in an intramedullary nail in a bone or similar object within a body of tissue having a depth. The system comprises an intramedullary nail targeting device, as described in the preceding paragraph, and an intramedullary nail, comprising at least one locking screw opening traversing the intramedullary nail, a magnet in association with the opening wherein the sensor array detects the magnetic flux lines of the magnet.

The present invention is also directed to a method for detecting the location and position of interlocking transverse screw openings within an intramedullary nail for the internal fixation of long bones within a limb, wherein the intramedullary nail includes a longitudinal opening and interlocking screw openings. The steps include placing the intramedullary nail in the marrow of the bone, wherein the intramedullary nail includes a magnet positioned at a reproducible distance from the opening; positioning an intramedullary nail targeting device, described above, near the general location of the opening, inserting the drill sleeve of the device in the limb such that the sensor foot touches the surface of the bone; activating the device to zero the sensor array; and positioning the device such that the display means determines the correct orientation of the device with respect to the bone for drilling.

The primary advantage of the current system is accuracy and the ability to use a magnet of considerably less strength. In addition, because the sensing array is now very close to the magnet, it is much more accurate.

Other advantages of the device of the present invention are as follows:
At least parts of the device are disposable and thus not subject to reuse which can cause health issues.
The device is conveniently handheld and portable.
The device is not affected by environmental metal or electrical interference.
The device can be used in any plane.
The device can self-zero and self-adjust to the strength of the targeting magnet.
The device can be used with cannulated or non-cannulated nails.
The sensor foot is small enough to be used percutaneously.

The objects and advantages of the invention will appear more fully from the following detailed description of the preferred embodiments of the invention made in conjunction with the accompanying drawings.

### BRIEF DESCRIPTION OF THE DRAWINGS

FIG. 1 is a perspective view of the IMN targeting device of the present invention.

FIG. 2 is a cross-sectional view of the IMN targeting device of FIG. 1 taken along lines 2-2 of FIG. 1.

FIG. 3 is a cross-sectional view of the foot of the IMN targeting device of FIG. 1 taken along lines 3-3 of FIG. 2.

FIGS. 4A and 4B are partial side plan views of the IMN targeting device of FIG. 1, illustrating an alternative embodiment of the sensor foot of the present invention.

FIG. 5 is a side plan view of the IMN targeting device illustrating its operation with respect to a long bone.

FIG. 6 is a top view of the intramedullary nail of the present invention.

FIG. 7 is a top plan view of the IMN targeting device of FIG. 1 with the cover (i.e., upper body portion) removed.

FIG. 8 is a block diagram illustrating the operation of the IMN targeting device of the present invention.

FIG. 9 is a top plan view of the IMN targeting device of FIG. 1 illustrating the display window.

FIG. 10 is a diagram illustrating the amplitude output of the sensors.

FIG. 11 is a diagram illustrating the flux density of density of the sensors.

### DETAILED DESCRIPTION OF THE NEW EMBODIMENT TO THIS INVENTION

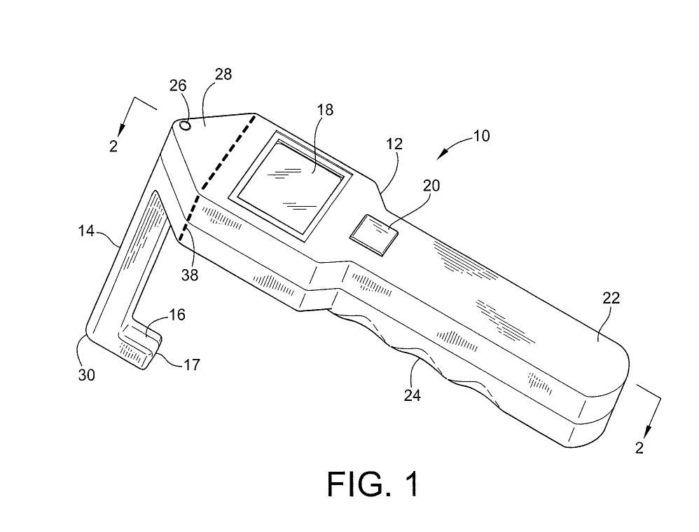

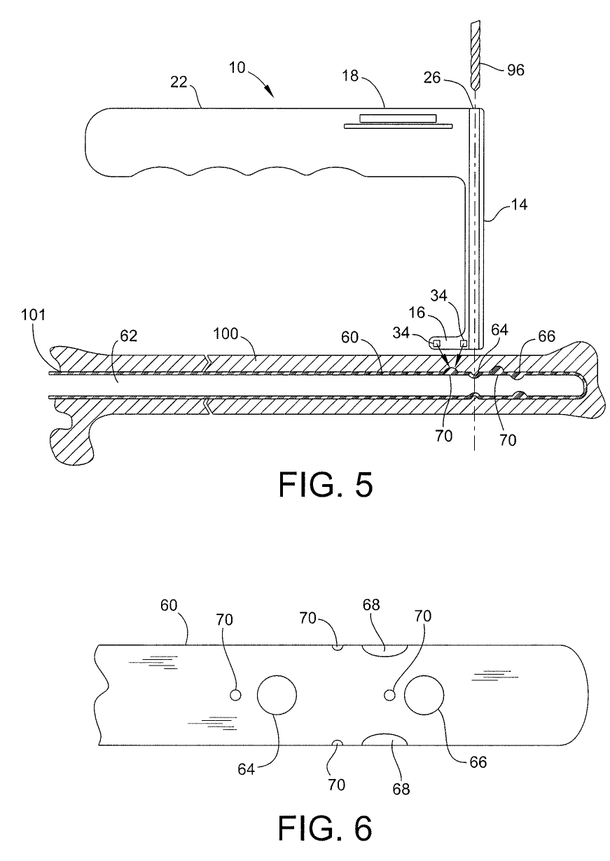

Referring now to FIG. 1, the present invention is directed to an IMN targeting device 10 which includes a body 12 with a handle portion 22, a drill sleeve 14, an activation or on/off button 20, a sensor foot 16 connected to the distal end of the drill sleeve 14, a display window 18, and a drill guide 26 extending through the interior of the drill sleeve 14. Advantageously, the IMN targeting device 10 places the sensor foot 16 of the drill sleeve 14 directly on the bone 100, illustrated in FIG. 5, for more accurate reading.

### Body 12

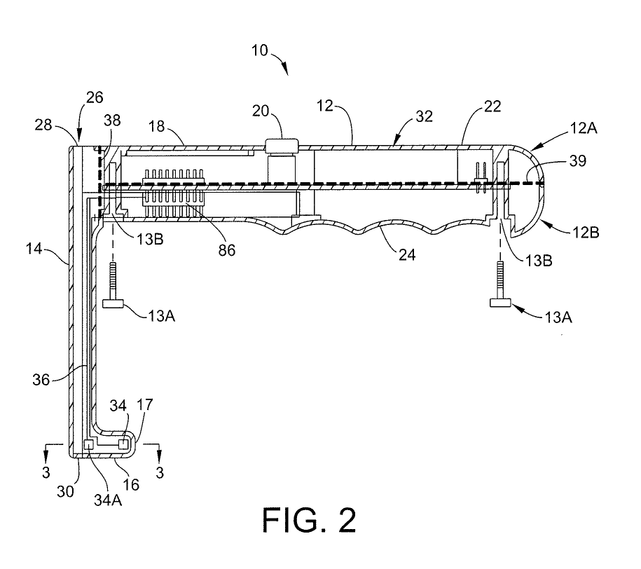

The body 12 can be made of a variety of materials known to the medical arts, including plastic and metal as appropriate for durability and reusability of the device 10. As illustrated in FIG. 1, the body 12 is designed to be handheld and comfortable with finger grips 24 in the handle 22. The body 12 also holds the batteries 32, the electronic process circuits 86 and the display window 18 as illustrated in FIGS. 2, 5 and 7. Typically, the device 10 can operate on two AAA batteries. Alternatively, the battery system can be rechargeable cells or the device 10 could be wired for electrical operation.

The body 12 of the device 10 is amenable to several non-limiting, non-mutually exclusive design variations, each with various advantages. First, the body 12 and sensor-drill sleeve 14 may be provided as separate units and may be separable, for example, at line 38 (see FIGS. 1 and 2). Connecting elements are known to the art for joining the drill sleeve 14 to the body 12 in a manner to enable the electrical connection between the two units. The body 12, which contains the electronic circuitry (such as the comparator circuit module 86), could be placed in a sterile bag (not illustrated) and would not have to be sterilized prior to use. During use, the plastic bag containing the body 12 could be perforated by the sensor-drill sleeve portion 14 of the device to connect to the electronic circuitry in the body 12 to render the device 10 ready for use. Having the drill sleeve 14 and the body 12 as separate units also allows for different interchangeable sensor-drill sleeve 14 options for the same body 12. The advantage of having different sensor-drill sleeve 14 combinations is that they can be used for different applications such as humeral or tibial nail-locking, which might use smaller diameter locking screws. Additionally, shorter drill sleeves 14 would allow more efficient use of the device 10 when deep soft tissues do not have to be avoided. The ability to use different sensor-drill sleeve 14 combinations therefore prevents the necessity of making a different targeting device 10 for each application. Finally, the separate drill sleeve 14 can be made of disposable materials for simple disposal after use.

In a second design variation, the electronics can be gas sterilizable so that the drill sleeve 14 section could be attached to the body 12 at line 38 and used without a sterile bag. All of the advantages above would be realized.

In a third design variation, the electronics could be made to withstand any other form of sterilization: autoclaving, CIDEX® disinfecting solutions (Johnson & Johnson Corporation, New Brunswick, N.J.) or similar chemical soaks, gas sterilization or any equivalent.

In a fourth design variation, the targeting device 10 can be connected wirelessly between the sensor foot 16 and the display window 18 to transfer targeting or display information wherever needed. The sensing information could be transmitted by radio, infrared or equivalent from the sensor handle to the display window 18. The display window 18 may be separate from the body 12 and can comprise any medium, including virtual projections, heads-up glasses, or a personal computer or television screen. Such a display window 18 can be made from any compatible non-magnetic material.

In a fifth design variation, the body 12 may be separable along line 39, shown in FIG. 2, to divide the body 12 into an upper body portion 12A and a lower body portion 12B. The upper and lower body portions 12A and B, may be connected by screws 13A that insert into threaded holes 13B, the latter of which extend from the lower body portion 12B into the upper body portion 12A. Other means of connecting the upper and lower body portions 12A and B may be used. The ability to separate the upper and lower body portions 12A and B allows the user to access internal parts of the device 10, such as the battery 32 and the comparator circuit 86.

### Window Display 18

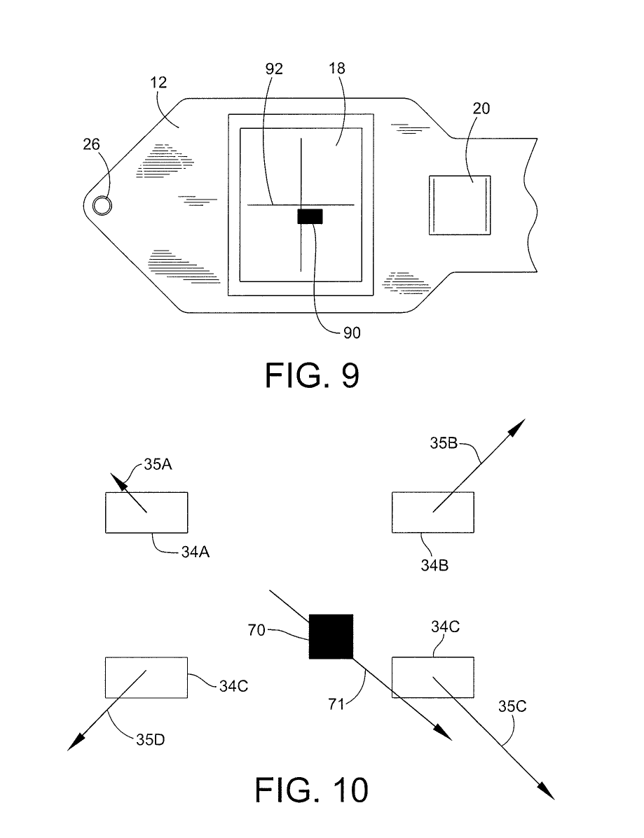

While the display window 18 can operate in the manner described with respect to Szakelyhidi et al., the display window 18 is preferably graphical in nature and provides a crosshair 92 in combination with a moving icon or target dot 90, illustrated in FIG. 9, to indicate the amount of misalignment of the sensor array 34 with respect to the magnet 70 on the IMN 60. Referring to FIGS. 5 and 9, when the targeting dot 90 is centered on the crosshair 92, the drill guide 26 is centered over the opening 64 or 65 in the IMN 60, and the bone 100 may be drilled through the drill guide 26. An advantage of this type of display is that it has sub-millimeter resolution. The determination of the “targeter centered” condition is entirely up to the surgeon, and does not depend on software in the device 10 to provide a “green light, drill here” indication. Such an indication would have to be determined by a software engineer who typically has no medical experience, and this is inappropriate at best. Although the device 10 may include such program logic, the device 10 preferably does not include such program logic because it would replace to some extent the judgment of the surgeon.

### Activation or On/Off Button 20

The activation button 20 is provided generally on the top surface of the body 12 at a convenient location for the surgeon to power and calibrate the device 10. The button 20 is positioned for comfortable use. There may be a button 20 on either side of the handle 22 activating the same function, to allow for left- or right-handed use.

### Drill Guide 14 and Sensor Foot 16

The preferred design of the present invention includes a drill sleeve 14 about 10 cm in length. While the length of the drill sleeve 14 is variable, a length of 10 cm incorporates most distal femoral soft tissue sleeves. For tibial and humeral applications, the drill sleeve 14 can be as short as 3-4 cm.

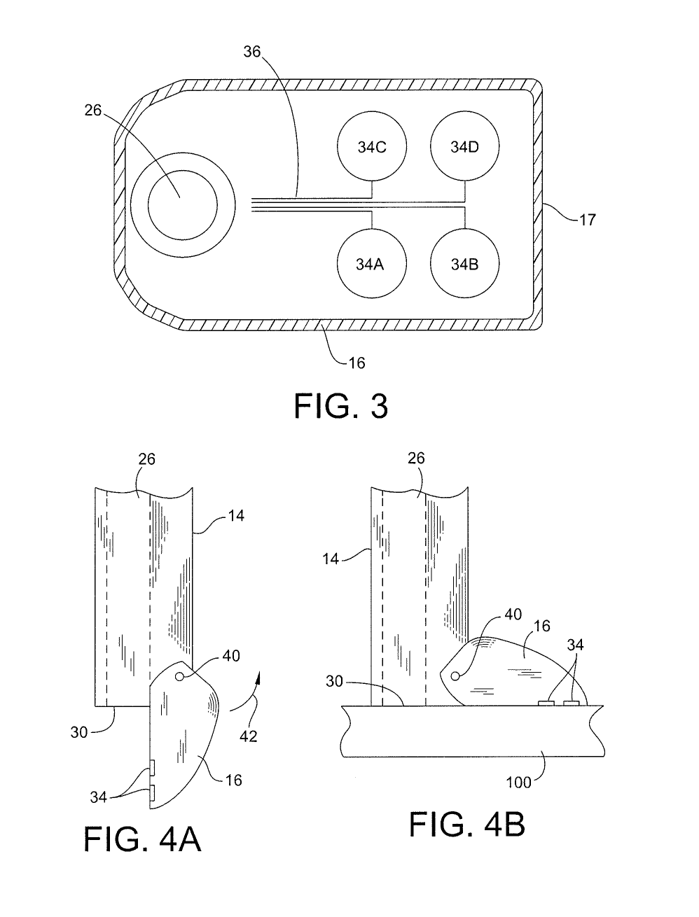

The sensor foot 16 is incorporated as part of the molded drill sleeve 14. The sensor foot 16 resembles a foot wherein the toe portion 17 contains the system of sensors 34A, B, C, D, as illustrated in FIG. 3. A smaller sized sensor foot 16 on the drill sleeve 14 is more practical to use. Alternatively, the sensor foot 16 could be made separate and incorporated into a metal drill sleeve 14.

In an alternative version of the sensor foot 16, as shown in FIGS. 4A and 4B, the sensor foot 16 can be a swivel design wherein it is hingedly attached to the drill sleeve 14 by means of a hinge unit 40. This configuration eases insertion of the sensor foot 16 into the soft tissues at the point of insertion. The hinge unit 40 can be made of a number of materials and designs to incorporate the swivel functioning of the unit. Prior to insertion into an opening in a limb for positioning next to a bone 100, the sensor foot 16 is rotated by means of the hinge 40 and pointed in parallel alignment with the drill sleeve 14 for ease of movement toward the bone 100, as illustrated in FIG. 4A. As the toe portion 17 comes in contact with the bone 100, the foot 16 will rotate in an arc approximating arrow 42 until the foot portion 16 rests on the bone 100 approximately perpendicular to the drill sleeve 14, as illustrated in FIG. 4B.

### Sensor Array 34

As illustrated in FIG. 3, the sensor foot 16 is preferably fitted with four magnetic sensors 34A, B, C, D arranged in a generally rectangular sensor array. The sensor array 34 is connected to the main electronics 86 in the body 12 by printed circuit wiring or wires 36 extending within the drill sleeve 14 beside the drill guide 26 (see FIG. 2). It is within the scope of the present invention to use different magnet shapes and materials as long as the sensor array 34 used to target them is adjusted to match the flux field of the magnet. It must also provide the desired flux field for feedback of discriminate targeting in all required planes. An electro-magnet may be used to achieve a similar field if desired. A preferred example of a magnet which may be used in the sensor array 34 is a Honeywell HMC 1052 (Morristown, N.J.) magneto resistive magnet. Magneto resistive magnets advantageously have an internal magnetic reset function that can reverse the magnetizing effect of a permanent magnet when brought too close to the sensor array 34. This feature works well and is used to reset the sensors 34 upon every calibration operation (described below). The sensor reset driver pushes a large current pulse through all sensors at once to perform the reset.

Essentially the same sensor array 34 as described with respect to Szakelyhidi et al. is used in the present invention to indicate the correct placement of the drill guide 14 over the IMN 60. However, rather than requiring eight sensors as suggested in Szakelyhidi et al., it is within the scope of the present invention to employ fewer sensors, e.g., four sensors 34A, B, C, D. The sensors 34A, B, C, D are affixed directly on the sensor foot 16 of the drill sleeve 14 at the area of the drill guide 26.

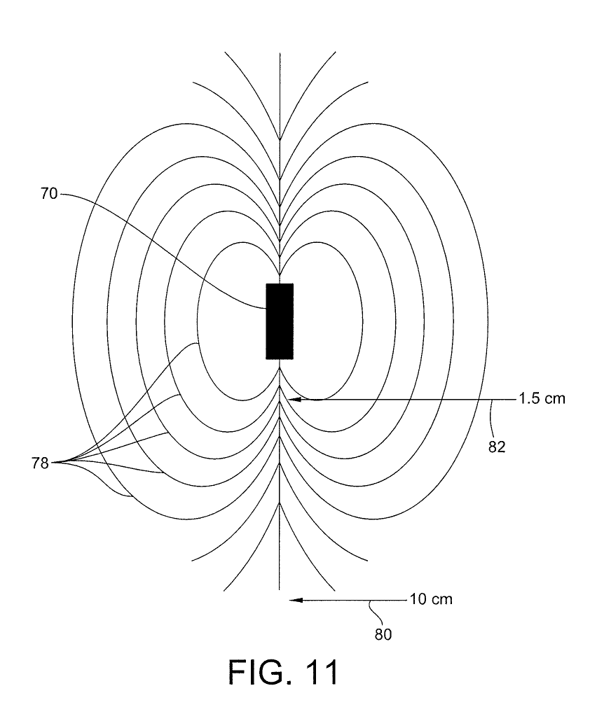

The sensors 34A, B, C, D are preferably sized and configured such that, at 10 cm 80, the sensor array 34 detects one or fewer flux lines 78 at a time, as shown in FIG. 11. Only by positioning the sensors 34 very near to the magnet 70 on the surface of the IMN 60 can the flux density be translated into targeting information by detecting multiple flux lines 78 (see reference 82 in FIG. 11). Placing the sensor array 34 on the foot 16 allows the sensor array 34 to be at the surface of the bone 100 in close proximity to the magnet 70. As illustrated in FIG. 11, the very high flux density at the surface of the bone 100 (see reference number 82) ensures that each sensor 34 detects multiple flux lines 78 in spite of the relatively small size of the sensors 34. This allows for greater resolution in targeting. As a non-limiting example, the sensors 34A, B, C, D are preferably 1-2 mm square and are arranged in an array 34 about 5-8 mm across and 2-5 mm thick. The center of the magnetic field can be as little as 6-10 mm from the center axis of the hole to be drilled. Targeting will be more accurate when the distance from the sensor magnet 70 to the center axis of the hole 64 or 66 in the IMN 60 is minimized.

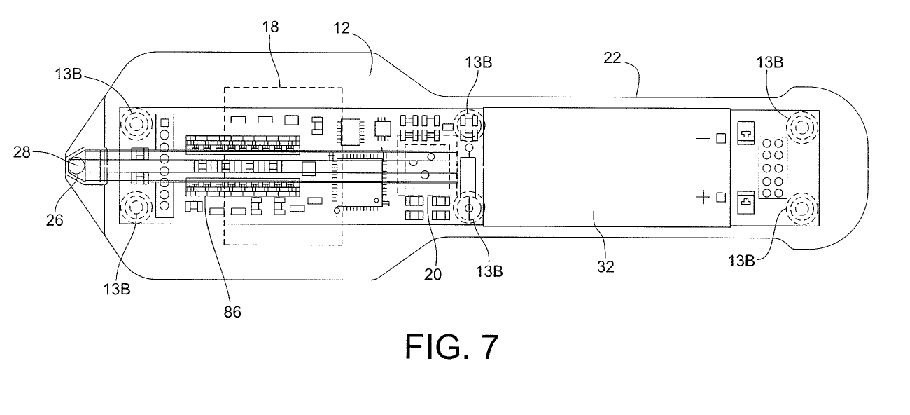

The sensor array 34 may be molded into a plastic drill sleeve 14 with the wires 36 from the sensor 34 ascending the drill sleeve 14 to the comparator circuit 86, as linked to an LCD window display 18, as illustrated in FIGS. 2 and 7.

### Intramedullary Nail 60

Referring to FIG. 5, the device is illustrated in association with a long bone 100, such as a broken femur, tibia or humerus bone. Within the bone 100, there is illustrated an intramedullary nail (IMN) 60, known to the art. Examples of IMNs are prevalent in the prior art. For example, reference is made to U.S. Pat. No. 6,503,249 to Krause and the patents to Durham (cited herein), the contents of which are incorporated herein for a description of IMNs and manners of use. The IMN 60 is an elongated metal rod typically having a hollow body portion or shaft 62, although, as described with respect to the IMN 60 in FIG. 6, the IMN 60 may also be a solid body. The IMN 60 typically includes a first locking screw opening 64 and a second, more distal locking screw opening 66. While the screw openings 64, 66 of typical IMNs 60 are transverse, i.e., positioned at a ninety degree angle in relation to the long axis of the nail 60, as illustrated in FIGS. 5 and 6, it is within the scope of the present invention to have non-transverse or oblique screw openings, i.e., openings at other than ninety degrees in relation to the length of the IMN 60. It is also within the scope of the present invention to have screw openings 68 placed along the circumferential axis (e.g., 90 degrees) relative to screw openings 64, 66, as illustrated in FIG. 6. Prior to placement of the IMN 60, a reaming rod known to the art is worked through the medullary cavity 101 of a long bone 100, such as a broken femur, tibia or humerus bone. The IMN 60 is then placed within the medullary cavity 101 for securing within the bone 100 by means of cross-locking screws or bolts positioned through the screw openings 64, 66.

### Magnet 70

Reference is made to Szakelyhidi et al. for a complete description of the magnetic field and its use with respect to the present invention. All magnets obey the inverse square rule, where the strength of the magnetic field drops off at the square of the distance. For example, doubling the distance decreases the magnetic field strength by 25%. If the distance is 10 cm, the magnetic field is 0.01 times (1%) the strength and field density it would be at 1 cm from the magnet. Conversely, the strength of the magnetic field at 1 cm from the magnet would be 100 times stronger than the same magnetic field measured at 10 cm. Early magnetic sensors were about 5 mm in size and prevented construction of a foot plate of practical size. New magnetic sensors are as small as 1-2 mm and make practical the construction of a magnetic sensing drill sleeve for placing directly on the bone.

As illustrated in FIGS. 5 and 6, the IMN 60 advantageously has the magnet 70 embedded directly on the surface of the IMN 60. Therefore, the IMN 60, as illustrated in FIG. 6 can be a solid IMN. It is also within the scope of the present invention to feed a removable magnet through the center of the IMN 60 as disclosed in Szakelyhidi et al. It is also within the scope of the invention to place a magnetic ring around the periphery of the opening 64, 66 or to place the magnet 70 in the center of the opening 64, 66, as a displaceable “bull's-eye.” The magnet 70 can be alternatively located at the opening 64, 66 and be on a swivel that retracts when the drill enters the opening 64, 66 of the IMN 60. In thin-wall IMNs, the magnet is centered within the IMN by a circular spring mechanism or equivalent. In thick-wall IMNs, the magnets are small enough to be within the diameter of the guide wire cannulation in the IMN.

### Flux Density

With the magnet 70 residing on or near the surface of the IMN 60, there is a close positioning of the sensor array 34 and the magnet 70 as shown in FIG. 5. This permits exposure of the sensor array 34 to greater flux density (see FIG. 11). All magnets, such as magnet 70, obey the inverse square rule, i.e., double the distance and the magnetic field is one-fourth the strength. As described above, if the distance between the sensor array 34 and the magnet 70 is 10 cm, the magnetic field is 1% the strength and field density of a sensor array 34 1 cm from the magnet 70. A preferred distance between sensor array 34 and the magnet 70 in the present invention is a distance of about 1.5 cm, typically the average thickness of the side of the bone 100. At that distance, the field density is about 30 times the density at a distance of 10 cm.

As illustrated in FIG. 11, a magnetic field sensed at 10 cm (reference number 80) has spread so thin that the flux lines 78 are too far apart to accurately locate the center of a 5 mm hole. At about 1.5 cm from the magnet (reference number 82), the flux density is high enough to detect sufficient positional information for accurate targeting of the device 10.

To date the most difficult distal targeting goal has been the distal femur. The working distances from the center line of the IMN 60 is typically no more than 3 cm at the surface of the distal femur and is usually 1-2 cm. This makes targeting nearly any other bone: tibia, humerus, or any other long bone even easier because of smaller cortex to nail distances.

### Internal Operation of Device 10

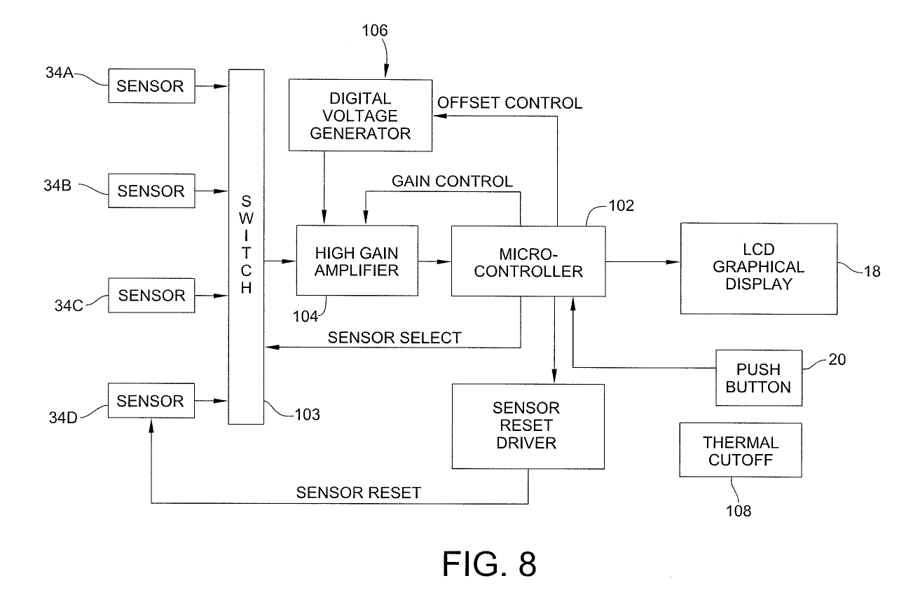

Reference is now made to FIGS. 7 and 8 for a description of the internal operation of the device 10. In action, the microcontroller 102 powers a single sensor 34A, B, C, or D in turn, using the switch 103 to connect it to the high gain amplifier 104. The microcontroller 102 then sets the digital voltage generator 106 to a predetermined value. The microcontroller 102 waits for the sensor 34A, B, C, or D and amplifier 104 to settle and then reads the voltage from the amplifier 104. This voltage is proportional to the applied magnetic field but also contains some environmentally generated noise and noise which is inherent in the sensors 34A, B, C, or D. The microcontroller 102 selects the four sensors 34A, B, C, or D in sequence, measuring their outputs and saving them for targeting computations. A complete set of measurements is made typically 20 to 50 times per second. As with any high gain sensor system, small errors can be multiplied by factors of 1000 or more, resulting in huge problems making the required measurements. The sensors 34A, B, C, or D are no different and have offset errors in their outputs that make measurements difficult without some adjustment. The amplifier 104 introduces errors as well. The digital voltage generator 106 is used during the calibration process to null out these errors.

When the device 10 is powered on by the activation button 20, the device 10 immediately begins a calibration sequence. This involves selecting each sensor 34A, B, C, and D in turn and determining the value from the digital voltage generator 106 that is required to bring the amplifier 104 into its linear amplifying region of operation. This operation takes only a couple seconds. Thereafter, as each sensor 34A, B, C, and D is selected, the digital voltage generator 106 is loaded with the particular value for that sensor 34A, B, C, or D, resulting in nullification of static errors for that sensor's measurement. The circuit also features a two-step amplifier gain selection, though the software may use only the high gain setting. Such a system allows use of the device 10 for various thicknesses of human bone 100 without software changes. This design uses one amplifier 104 and an inexpensive commodity solid state switch 103 to select which sensor 34A, B, C, or D to read. Another feature not shown is that the microcontroller 102 does not leave all sensors 34A, B, C, or D powered continuously, but rather turns them on in sequence, saving power consumption.

The microcontroller 102 uses a vector algorithm to determine how to position the target icon 90 on the window display 18. The position of each sensor 34A, B, C, or D is assigned a vector direction depending on its position in the array 38. The amplitude of the output of each sensor 34A, B, C, or D provides the magnitude of each vector. Addition of the magnitudes of the vectors provide a resultant vector that determines the position of the device 10 relative to the magnet, which is represented as a two-dimensional position of a targeting dot 90 on the window display 18 (see FIG. 9). FIG. 10, for example, shows a center box representing the magnet 70 and four other boxes representing the magnetic sensors 34A, B, C, or D. The vector lines 35A, B, C, and D attached to each sensor 34A, B, C, and D, respectively, indicate the strength of the field at each sensor. Vector line 71 is the resultant vector, which indicates the direction the sensor array 34 should be moved to center it over the magnet 70. The magnet 70 in FIG. 10 corresponds with the targeting dot 90 in FIG. 9.

Referring back to FIG. 8, the thermal cutoff 108 is present in case the device 10 is accidentally run through a sterilizer cycle. The thermal cutoff 108 activates at 82° Celsius and disables operation of the device 10 permanently. Without the thermal cutoff 108, it is likely that the device 10 would work somewhat after being exposed to such heat, but reliable operation could not be guaranteed. A low battery indicator is implemented that warns the user of low batteries 32 on the window display 18 and also prevents the device 10 from operating.

### User Operation

The single activation button 20 is used to turn the device 10 on, and the device 10 immediately performs a calibration cycle. If the button 20 is pressed briefly thereafter, another calibration cycle is initiated. The window display 18 indicates to the user that calibration is in progress. It is not possible to turn the device 10 on without initiating a calibration cycle. To turn the device 10 off, the button 20 is held down for a couple seconds until the display goes off. The device 10 also powers off after two minutes to prevent the batteries from draining.

To perform targeting, the device 10 is held in the same orientation as it will be used. The device 10 is raised 10-12 inches above the targeting magnet 70 and the button 20 is pressed to start a calibration cycle. It is important that the device 10 be oriented approximately as it will be used in order to properly null the magnetic field of the earth. Once the device 10 completes its calibration operation, it is lowered to the work area and moved to achieve an on-target indication.

### Performance

If the device 10 has some difficulty detecting the magnetic field of the targeting magnet 70, the display 18 will show an error indication. There could be reasons for the error indication:
The magnetic field of the earth: This field is about 0.5 gauss. The device 10 must be used with a targeting magnet 70 and at a distance such that the earth's magnetic field is not a significant factor.
Alternating current (AC) power and radio frequency (RF) noise sources: These are minimized through device shielding.
Sensor noise: The sensors 34A, B, C, D produce electronic noise as a byproduct of their operation. This noise is added to the voltage output of each sensor caused by the magnetic field. The device 10 must be used with a targeting magnet 70 and at a distance such that the sensor noise is not a significant factor.
Proximate ferromagnetic objects: The earth's magnetic field bends where it approaches magnetically attractive objects, such as chairs, metal tables, rebar in concrete, etc. However, if the earth's magnetic field is not an issue, then proximate ferromagnetic objects will not be an issue, either.
   Placing the sensor array 36 proximal to the magnet 70 (e.g., against the bone), as occurs with the present device 10, successfully avoids the above problems by ensuring a strong magnetic field at the sensors when the device is in use.

### Operation of Device 10

In order to locate the general location of the openings 64 and 66 in the IMN 60, the IMN 60 is placed in the marrow of the bone 100 and urged through the bone 100 as described in Szakelyhidi et al. The openings 64, 66 in the IMN 60 to be targeted has a magnet 70 placed at a reproducible distance from the openings 64, 66 with the magnetic field oriented to the magnetic sensor array 34 in the foot 16 of the IMN targeting device 10. A handle wand extension, known in the art, which is the same length as the IMN 60 and attached to the IMN 60, is urged over the exterior of the limb along the same direction as the IMN 60.

When the IMN 60 is fully positioned in the bone 100, the end of the handle wand extension indicates both the end of the IMN 60 and the approximate location of the openings 64, 66 in the IMN 60 in the bone 100. The magnet 70, situated on the surface of the IMN 60, as illustrated in FIG. 6, corresponds to the distance of the openings 64, 66. Alternatively, a magnet can be placed in the hollow shaft of the IMN 60, carried down the cannulation of the IMN 60 by a handle wand, and locked at a corresponding distance of the opening 64 or 66 to be targeted or placed into the wall of the IMN 60.

An incision is made in the limb. An oval trochar can be used to make a path for the drill sleeve 14 down to the surface of the bone 100. Once placed on the surface of the bone 100, the display window 18 is activated by the action of the on/off button 20. A signal is sent to the sensor array 34 to zero the sensors 34A, B, C, D. When the sensor array 34 is moved across the surface of the bone 100, the sensor information appears on the display window 18, generally in the form of a targeting dot 90 on a targeting grid 92 as illustrated in FIG. 9. The placement of the targeting dot 90 in the center of the targeting grid 92 indicates correct placement of the device 10 for drilling. In addition to moving the targeting dot 90 centrally with respect to the targeting grid 92, more accurate information could be attained in the form of enlarging the dot 90 in response to the strength of the magnetic field being sensed. FIG. 9 illustrates the device 10 wherein the targeting dot 90 is misaligned with respect to the targeting grid 92, indicating that the drill sleeve 14 placement is incorrect with respect to the IMN 60 within the bone 100. Once the targeting dot 90 is centered on the targeting grid 92, this “maximum size” ensures that the targeting device 10 has not been sensing a symmetrical set of field lines around the magnet 70 or a flux pattern created between two or more magnets 70 embedded into the side of a solid IMN 60. The same information could be displayed in any equivalent fashion such as a variable LED, audio output, color change or similar signaling device.

Typically, the drill bit 96, illustrated in FIG. 5, used in distal targeting drills the minor diameter of the screw to be inserted in the IMN 60. This gives more room for the targeting to be accurate than if the major diameter were to be drilled first. A star point drill prevents the drill from “walking” on the slippery curved surface of the bone and is therefore preferred.

The drill bit 96 is then inserted into the drill guide 26 at the upper drill sleeve opening 28 while this information is obtained. The lower opening 30 of the drill guide 26 is placed directly on the bone 100. This is accomplished by affixing the sensors 34 directly on the drill guide 26 at the area of the opening 64 or 66. The drill sleeve 14 is inserted into the bone 100 at the location of the opening 64 or 66.

The sensor array 34 is activated to locate the magnet 70, which then determines the location of the opening 64 or 66. The information sent to the comparator circuit 86 is processed and displayed on an LCD screen 106 that moves a target dot to the center of a cross hair that represents the center of the magnetic field of the targeting magnet.

The drill 96 with a star point is located adjacent to the target magnet 70 and is parallel to the IMN 60 when the magnetic sensor 34 is balanced over the magnetic field. Because the sensor array 34 is proximal to the openings 64 or 66, when the field is balanced, the drill 96 is free to pass through the opening 64 or 66 in the IMN 60 to the opposite cortex. As soon as the target dot 90 aligns at the center of the targeting grid 92, the drill 96 is drilled through the opening 64 or 66 to the opposite cortex. The process could be repeated for any additional holes to be targeted.

To target openings 68 in the IMN 60 as shown in FIG. 6, the magnetic wand can be rotated 90 degrees. If the magnets 70 are implanted in the side wall of the IMN 60, they are available for targeting even after locking the IMN 60 proximally.

An aiming device is always more accurate if it has two references in space to align it. The first reference to assist the accuracy of the device 10 comes from determining an entry point on the skin directly over the opening to be targeted in the IMN 60. The easiest way to determine this point is with a wand that extends from the handle that holds the IMN 60 for insertion. The wand reproduces the curvature of the IMN 60 and has markings corresponding to the length of each IMN 60. The wand shows the correct entry point over each opening so that when the drill sleeve 14 is inserted at that point, the soft tissues help to stabilize the device perpendicular to the axis of the IMN 60. The importance of being able to rest the device 10 on the surface of the bone 100 during use cannot be over emphasized. The accuracy needed is on the order of 1 mm. A device 10 held in space cannot be as accurate while simultaneously using a drill.

In most applications it is advantageous to insert the screw through the lumen in the cannula after the opening in the IMN 60 has been magnetically targeted. The device 10 would be used in the standard fashion to drill the minor diameter of the locking screw. A calibration on the drill measures the depth of the drilled hole at the upper drill sleeve 14 opening 28 of the device 10. Alternatively, after drill removal, the device 10 can remain against the bone 100. When the drill guide 26 is removed, a depth gauge is used to measure the length of the screw to be inserted. Once measured, the screw of the appropriate length is loaded onto a screw driver and inserted across the opening 64, 66 of the IMN 60. Self tapping screws are used in the preferred embodiment.

It is understood that the invention is not confined to the particular construction and arrangement of parts herein illustrated and described, but embraces such modified forms thereof as come within the scope of the following claims.

I claim:

1. An intramedullary nail targeting device for detecting the precise location and position of an opening in an intramedullary nail in a bone or similar object within a body of tissue having a depth, comprising:
a. a body comprising a handle end, a drill sleeve end, a power source and processing circuits for sensing the correct orientation of the device with respect to the bone;
b. an activation button;
c. an extended drill sleeve connected to the drill sleeve end and having a first proximal end and a second distal end, wherein the drill sleeve has a length necessary to extend through the depth of the tissue, the drill sleeve including:
i) a sensor foot at the distal end of the drill sleeve, and
ii) a drill guide extending from the proximal end of the drill sleeve to the distal end, and
iii) a sensor array within the sensor foot for sensing the opening in the intramedullary nail when the sensor foot is placed on or near the surface of the bone; and
d. display means to determine the correct orientation of the device with respect to the bone when the sensor foot is placed on or near the surface of the bone.

2. The device of claim 1 wherein the display means is an LCD display window.

3. The device of claim 1 wherein the display means is a remote projector.

4. The device of claim 1 wherein the power source is selected from the group consisting of batteries, rechargeable cells, or an electrical source.

5. The device of claim 1 wherein the body and the drill sleeve are one unit.

6. The device of claim 1 wherein the drill sleeve is releasably connected to the body.

7. The device of claim 6 wherein the drill sleeve is made of disposable material.

8. The device of claim 1 wherein the length of the drill sleeve corresponds with the depth of the tissue.

9. The device of claim 1 wherein the sensor array is connected to the processing circuits by wires extending the length of the drill sleeve.

10. The device of claim 1 wherein the sensor array is wirelessly connected to the processing circuits in the body of the device.

11. The device of claim 1 wherein the drill sleeve has a length of between about 3 cm and 12 cm.

12. The device of claim 1 wherein the drill sleeve has a length of about 10 cm.

13. The device of claim 1 wherein the sensor foot is fixedly attached to the distal end of the drill sleeve.

14. The device of claim 1 wherein the sensor foot is hingedly attached to the distal end of the drill sleeve.

15. The device of claim 1 wherein the sensor array comprises four magnetic sensors arranged in a generally rectangular array.

16. The device of claim 15 wherein the magnetic sensors are selected from the group consisting of electro-magnets and magneto resistive magnets.

17. The device of claim 15 wherein the magnetic sensors are magneto resistive magnets.

18. The device of claim 15 wherein the sensors are approximately 1-2 mm square.

19. The device of claim 1 wherein the generally rectangular array of magnetic sensors intersects at a center and wherein a center of the rectangular array is between about 6 and about 10 mm from the center axis of the opening in the intramedullary nail.

20. A system for detecting the precise location and position of an opening in an intramedullary nail in a bone or similar object within a body of tissue having a depth, comprising:
a. an intramedullary nail targeting device, comprising
i) a body comprising a handle end, a drill sleeve end, a power source and processing circuits for sensing the correct orientation of the device with respect to the bone;
ii) an activation button,
iii) an extended drill sleeve connected to the drill sleeve end and having a first proximal end and a second distal end, wherein the drill sleeve has a length necessary to extend through the depth of the tissue, the drill sleeve including:
(1) a sensor foot at the distal end of the drill sleeve,
(2) a drill guide extending from the proximal end of the drill sleeve to the distal end, and
(3) a sensor array within the sensor foot for sensing the opening in the intramedullary nail when the sensor foot is placed on or near the surface of the bone; and
iv) display means to determine the correct orientation of the device with respect to the bone when the sensor foot is placed on or near the surface of the bone; and
b. an intramedullary nail, comprising:
i) at least one locking screw opening traversing the intramedullary nail,
ii) a magnet in association with the opening wherein the sensor array detects the magnetic flux lines of the magnet.

21. The system of claim 20 wherein the magnet is embedded on the surface of the intramedullary nail.

22. The system of claim 20 wherein the magnet includes the following characteristics: a) a magnetic field of sufficient strength to match the sensor array of the device, and b) a magnetic field of sufficient shape to provide the desired feedback necessary to discriminate targeting in all required planes.

23. The system of claim 20 wherein the sensor array is positioned a distance of about 1.5 cm from the magnet to detect the magnetic flux lines of the magnet.

24. The system of claim 20 wherein the drill sleeve is releasably connected to the body.

25. The device of claim 20 wherein the drill sleeve has a length of about 10 cm.

26. A method for detecting the location and position of interlocking transverse screw openings within an intramedullary nail for the internal fixation of long bones within a limb, wherein the intramedullary nail includes a longitudinal opening and interlocking screw openings, comprising:
a. Placing the intramedullary nail in the marrow of the bone, wherein the intramedullary nail includes a magnet positioned at a reproducible distance from the opening;
b. Positioning an intramedullary nail targeting device near the general location of the opening, wherein the intramedullary nail targeting device comprises:
i) a body comprising a handle end, a drill sleeve end, a power source and processing circuits for sensing the correct orientation of the device with respect to the bone;
ii) an activation button,
iii) a extended drill sleeve connected to the drill sleeve end and having a first proximal end and a second distal end, wherein the drill sleeve has a length necessary to extend through the depth of the tissue, the drill sleeve including:
(1) a sensor foot at the distal end of the drill sleeve,
(2) a drill guide extending from the proximal end of the drill sleeve to the distal end, and
(3) a sensor array within the sensor foot for sensing the opening in the intramedullary nail when the sensor foot is placed on or near the surface of the bone;
iv) display means to determine the correct orientation of the device with respect to the bone when the sensor foot is placed on or near the surface of the bone;
c. inserting the drill sleeve of the device in the limb such that the sensor foot touches the surface of the bone;
d. activating the device to zero the sensor array; and
e. positioning the device such that the display means determines the correct orientation of the device with respect to the bone for drilling.
